(19) United States
(12) Patent Application Publication
(10) Pub. No.: US 2018/0011080 A1
(43) Pub. Date: Jan. 11, 2018

(54) DIAPERS WITH URINE DETECTION FUNCTIONALITY AND METHODS FOR DETECTING ANTIBIOTIC

(71) Applicant: Baby Care Development Technologies Corporation, City of Industry, CA (US)

(72) Inventor: Jie Xu, Harbin (CN)

(21) Appl. No.: 15/643,363

(22) Filed: Jul. 6, 2017

(30) Foreign Application Priority Data
Jul. 6, 2016 (CN) 201610528015.4

(51) Int. Cl. G01N 33/493; A61F 13/42; G01N 21/81; A61B 5/00; A61B 5/01; G01N 21/78

(52) U.S. Cl. CPC G01N 33/493; A61B 5/01; G01N 21/78; G01N 21/81; A61B 5/0008; A61F 13/42; A61B 5/6808; A61F 2013/424; A61F 2013/421

(57) ABSTRACT

A diaper, having urine detection functionalities, comprises a urine isolation layer, an absorbent core layer and an outer layer. A urine detection device is disposed in between the urine isolation layer and the absorbent core layer. The urine isolation layer has an open end embedded in a sleeve of the absorbent core layer. The urine detection device comprises a detection circuit and a test paper composite. The test paper composite includes: a trace element test paper, an antibiotic test paper and a urine routine test paper. Each test paper is made up of test strips for detecting different elements. Each test strip produces results according to a different color indicator scheme. The antibiotic test paper includes a specimen absorption pad, a specimen binding pad, a reaction membrane, and an absorbent pad. An antibiotic detection method is provided.

### CROSS REFERENCE TO RELATED APPLICATIONS

This application claims the benefit and priority of Chinese Patent Application No. 201610528015.4, filed on Jul. 6, 2016, which is incorporated herein by reference in its entirety.

### TECHNICAL FIELD

The invention relates to a diaper having a urine detection function, and a method for detecting antibiotics in urine.

### BACKGROUND

At present, the widespread use of baby diapers instead of traditional diapers has the advantages of convenience, but parents need to regularly check whether one needs to replace diapers, because when the replacement is not timely, soaked and/or soiled diapers will cause the burns and rashes on the skin of the baby. Improved diapers with urine display function, such as a diaper wet color suitable for hot melt adhesive for disposable sanitary products using methods such as those disclosed in Chinese Patent Application No. 201310469895.9, but with significant application usually diapers urine chemical principle, only after use of color to remind, parents still need regular observation. It is not very convenient for use at night.

A urine test research more than a thousand children of Jiangsu, Zhejiang, Shanghai from Fudan University, shows that 58% of children were detected with at least one kind of antibiotics, ¼ detected with more than 2 kinds of antibiotics, some samples even have 6 kinds of antibiotics, some antibiotics are commonly used antibiotics in animal husbandry. WHO (World Health Organization) has released that antibiotic resistant bacteria are spreading all over the world. (April 17th, reference news). Antibiotic resistance, like bad smog, will evolve into an environmental crisis that threatens everyone. Inappropriate use of drugs causes and intensifies changes in bacteria and other microorganisms, and eventually leads to ineffective use of drugs for the treatment of infections, which is the source of antibiotic resistance. The widespread use of antibiotics in animal husbandry can lead to the development of antibiotic resistance. Antibiotic resistance is spread in livestock and spreads through food borne diseases and other infections. A common example is the detection of commonly used antibiotics in children's urine. In the human body, the use of inappropriate drugs can also cause antibiotic resistance. There is no convenient way to test the antibiotic levels in children's urine.

At present, the detection of antibiotics in food field and environment field, method for the detection of antibiotic are high performance liquid chromatography, microbiological assay, immunoassay, these methods require expensive instruments or professional testing personnel, not suitable for family use, not for rapid detection of antibiotics in urine of children. The existing diapers cannot detect excrement such as urine, thereby analyzing the health status of users, etc., and further improvement is needed.

### SUMMARY

The technical problem to be solved is to provide a children's diapers, can have a urine detection function, including temperature, trace elements, antibiotics, and routine urine test, so as to obtain the user's health information.

In order to solve the technical problem, the disclosure provides a urine detection function of children's diapers. In some embodiments, the diaper comprises, from the interior to the exterior, a urine isolation layer, an absorbent core layer and an outer layer. A urine detection device may be disposed in between the urine isolation layer and the absorbent core layer. The urine isolation layer has an open end embedded in a sleeve of the absorbent core layer. The urine detection device may comprise a detection circuit and a test paper composite. The test paper composite includes: a trace element test paper, an antibiotic test paper and a urine routine test paper. Each test paper is made up of test strips for detecting different elements. Each test strip produces results according to a different color indicator scheme. The antibiotic test paper includes a specimen absorption pad, a specimen binding pad, a reaction membrane, and an absorbent pad. An antibiotic detection method is provided.

In some embodiments, the detection circuit of the diaper with urine detection function comprises: a controller, a temperature sensor communicatively coupled to the controller and configured to measure a temperature of a user, a humidity sensor communicatively coupled to the controller and configured to detect a humidity of the absorbent core layer, a wireless transmission module communicatively coupled to the controller and configured to communicate wirelessly with a remote mobile terminal, and a power supply module configured to provide power to the controller, the temperature sensor, the humidity sensor, and the wireless transmission module.

In some embodiments, the wireless transmission module may perform wireless communication according to the Bluetooth protocol or the WI-FI protocol.

In some embodiments, the test paper composite comprises a trace elements detection test paper for detecting trace elements, an antibiotic detection test paper for detecting antibiotics, and a urine test strip for detecting urine.

In some embodiments, each of the types of test paper includes one or more different test paper components for detecting different types of substances, each test component provides test results according to a different color indication scheme.

In some embodiments, each test component may have a length of 5-30 mm, a width of 5-20 mm, and a thickness of 1-5 mm.

In some embodiments, the urine detection test paper (urine test strip) performs at least one of the following testing or detecting: a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

In some embodiments, the diaper further comprises a specimen absorbing pad, a specimen binding pad, a reaction membrane, and a specimen liquid absorbing pad.

In some embodiments, the specimen liquid absorbing pad comprises a filter paper.

In some embodiments, the specimen binding pad is coated with at least a beta lactam specific antibody-colloidal gold marker, a tetracycline specific antibody-colloidal gold marker, a quinolone specific antibody-colloidal gold marker, a macrocyclic lipid specific antibody-colloidal gold marker, a sulfonamide specific antibody-colloidal gold marker, and a cephalosporin specific antibody-colloidal gold marker.

In some embodiments, the reaction membrane comprises a nitrocellulose membrane coated with a macrolide antibiotics detection line, a beta lactam antibiotics detection line, a tetracyclines antibiotics detection line, a quinolones antibiotics detection line, a sulfonamides antibiotics detection line, a cephalosporin antibiotics detection line, and a goat anti mouse control line.

In some embodiments, the macrolide antibiotics detection line and the beta lactam antibiotics detection line are disposed in parallel of each other on the reaction membrane, wherein the tetracyclines antibiotics detection line and the quinolones antibiotics detection line are disposed in parallel of each other on the reaction membrane, and wherein the sulfonamides antibiotics detection line and the cephalosporin antibiotics detection line are disposed in parallel of each other on the reaction membrane.

In some embodiments, the macrolide antibiotics detection line comprises macrolide antibiotic-carrier protein, wherein the beta lactam antibiotics detection line comprises beta lactam antibiotics-carrier protein, wherein the tetracycline antibiotics detection line comprises tetracycline antibiotics-carrier protein, wherein the quinolones antibiotics detection line comprises quinolone antibiotics-carrier protein, wherein the sulfonamide antibiotics detection line comprises sulfonamide antibiotics-carrier protein, and wherein the cephalosporin antibiotics detection line comprises cephalosporin antibiotics-carrier protein.

In some embodiments, each of the carrier proteins comprises bovine serum albumin.

The disclosure also provides a method for detecting antibiotics using a urine detection device arranged between the isolation layer and the absorbent core layer, wherein the urine detection device is connected with a wireless mobile terminal. The method comprises the steps of: scanning, by the mobile terminal, a code on the diaper to perform a pairing between the mobile terminal and the urine detection device of the diaper; monitoring, by the urine detection device, a humidity of the diaper based on a color change of a trace elements detection test paper of the diaper to determine if the diaper is wet; and in response to a determination that the diaper is wet, providing, by the mobile terminal, a prompt for changing diapers.

Preferably, the antibiotic detection method further comprises: determining, by the urine detection device, a temperature of the diaper, wherein the mobile terminal stores a normal temperature range; and in response to a determination that the temperature of the diaper is an abnormal temperature based on the stored normal temperature range, providing, by the mobile terminal, an alert.

In some embodiments, the antibiotic detection method of the invention, wherein determining the humidity of the diaper comprises: removing a urine isolation layer of the diaper and capturing, by the mobile terminal, an image of the trace elements detection test paper; detecting, by the mobile terminal, a color indication of the trace elements detection test paper; determining, by the mobile terminal, an abnormal condition based on a detection of a detection line on the diaper; and providing, by the mobile terminal, an alert in response to determining the abnormal condition.

The diapers according to various embodiments of the disclosure provide simple structure, low cost, can have a urine detection function, including temperature, trace elements, antibiotics, and routine urine test, so as to obtain users (children) of the health information, the mobile terminal can be combined with the use of long-term health monitoring of children. The utility model has the advantages of simple operation, no need of professionals, and can be used by families. The detection sensitivity is high, which can help to preliminarily determine the antibiotic content in the urine of children.

### BRIEF DESCRIPTION OF THE DRAWINGS

FIG. 1 illustrates a diaper with the urine detection function according to some embodiments of the disclosure;

FIG. 2 is a schematic diagram of a circuit for the urine detection device according to some embodiments of the disclosure;

FIG. 3 illustrates a diaper with a urine detection function having a code according to some embodiments of the disclosure;

FIG. 4 is a schematic diagram of a urine detection device when the urine isolation layer is removed from the diaper according to some embodiments of the disclosure; and

FIG. 5 is a flow chart of a urine detection method according to some embodiments of the disclosure.

Embodiments of the present disclosure and their advantages may be understood by referring to the detailed description herein. It should be appreciated that reference numerals may be used to illustrate various elements and features provided in the figures. The figures may illustrate various examples for purposes of illustration and explanation related to the embodiments of the present disclosure and not for purposes of any limitation.

### DETAILED DESCRIPTION

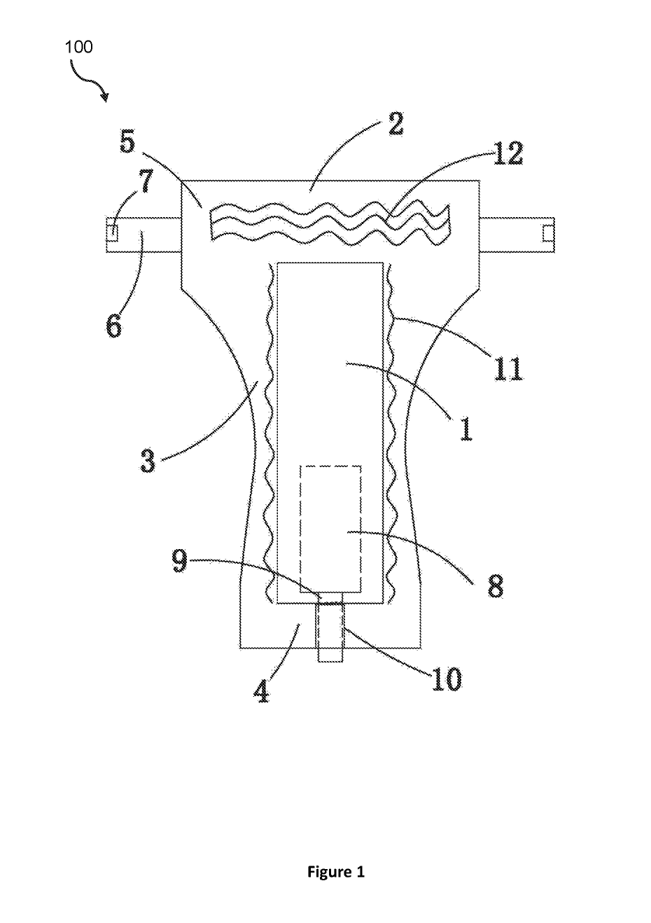

As shown in FIG. 1, the disclosure provides a diaper 100 (e.g., a baby diaper, an adult diaper, etc.) with urine detection functions. The diaper 100 includes absorbent core layer 1, rear waist portion 2, crotch portion 3, front waist portion 4; wing portion 5, adhesive tape 6, adhesive buckle 7, test paper composite 8, detection circuit 9, open end card sleeve 10 of the urine isolation layer, leakage prevention edge 11, and elastic band 12.

The diaper 100 includes, from interior to exterior, urine isolation layer, absorbent core layer 1, and the outer layer of the pants. The outer layer of the pants comprises rear waist portion 2, crotch portion 3, and front waist portion 4.

The two sides of the rear waist portion 2 include wing portion 5 and one end of adhesive tape 6. The other end of the adhesive tape 6 is a free end which is provided with an adhesive buckle 7. The urine isolating layer and the absorbent core layer 1 is provided with a urine detection device. The urine detection device comprises a detection circuit 9 and test paper composite 8. The urine isolation layer is provided with an open end that is embedded in the clamping sleeve 10 arranged in the absorption core layer 1. Inside the outer pants body is provided with a two contraction of 11 two flange leak, leak proof contraction of the flange 11 is located in the absorption core layer 1 on both sides, makes it keep in the baby liquid absorption core layer 1, no leakage from both sides. The inner side of the back waist 2 is transversely provided with an elastic contraction band 12, and both ends of the elastic contraction band 12 are fixedly connected with the two sides of the inside of the 2 sides of the back waist to prevent leakage of the toilet liquid.

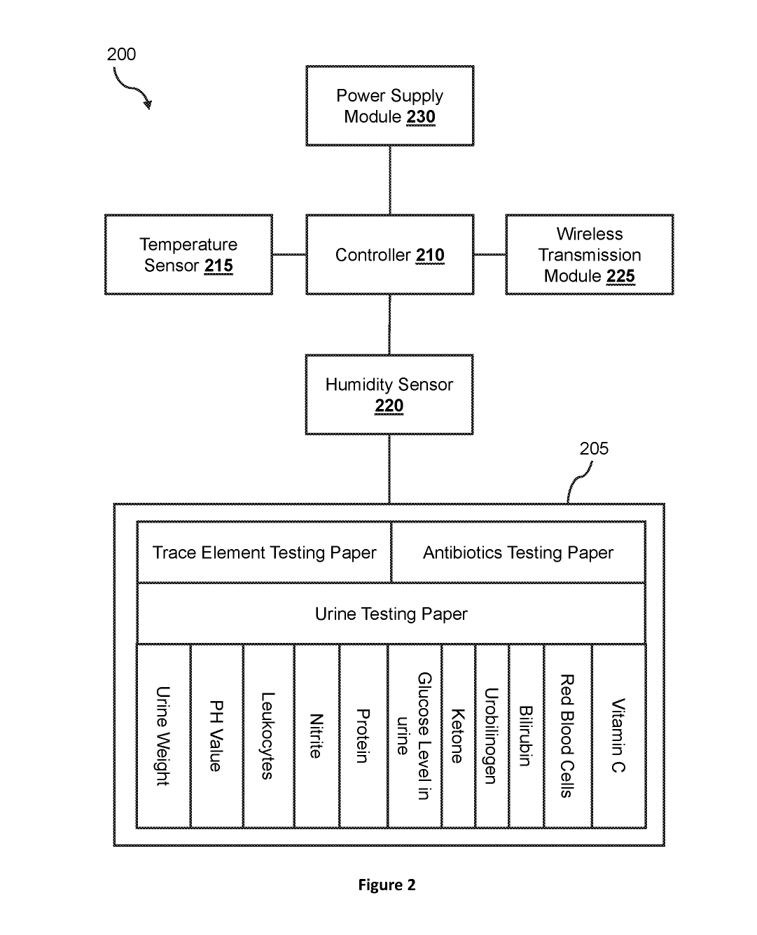

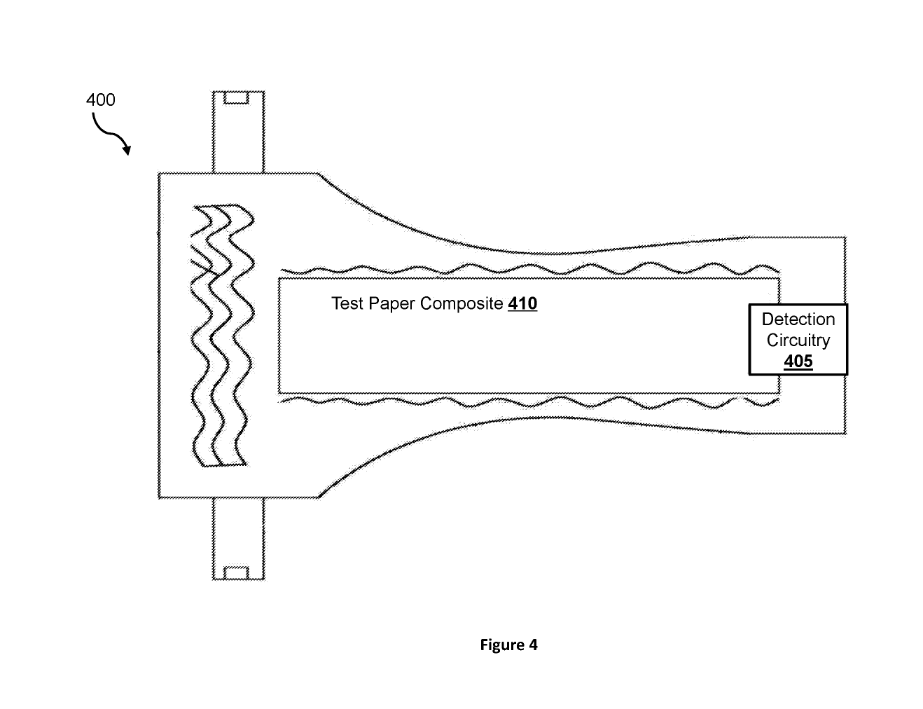

As shown in FIG. 2 and FIG. 4, the disclosed diaper includes a detection circuit. FIG. 2 illustrates an example detection circuit 200 and a test paper composite 205. the detection circuit of the diaper with urine detection function comprises: a controller 210, a temperature sensor 215 communicatively coupled to the controller and configured to measure a temperature of a user, a humidity sensor 220 communicatively coupled to the controller and configured to detect a humidity of the absorbent core layer, a wireless transmission module 225 communicatively coupled to the controller and configured to communicate wirelessly with a remote mobile terminal, and a power supply module 230 configured to provide power to the controller 210, the temperature sensor 215, the humidity sensor 220, and the wireless transmission module 225. In some embodiments, the wireless transmission module 225 may perform wireless communication according to the Bluetooth protocol or the WI-FI protocol.

FIG. 4 illustrates an example diaper 400 with a detection circuitry 405 and a test paper composite 410. In some embodiments, the test paper composite 410 is the same as test paper composite 205 of FIG. 2.

The disclosure discloses a diaper with the function of urine detection, wherein the test paper composite comprises a trace element test paper, an antibiotic detection paper and a urine routine test paper. Each of the trace element test paper, the antibiotic detection paper and the urine routine test paper may be made up of a plurality of test paper components for detecting different substances. Each of the plurality of test paper components may provide a test result according to a different color indication scheme. The test paper component for each test item may have a length of 5-30 mm, a width of 5-20 mm, and a thickness of 1-5 mm.

The urine detection test paper (urine test strip) performs at least one of the following testing or detecting: a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

The antibiotic test paper comprises a specimen absorbing pad, a specimen binding pad, a reaction membrane and a specimen water absorbing pad. The specimen water absorption pad comprises a filter paper.

In some embodiments, the specimen binding pad is coated with at least a beta lactam specific antibody-colloidal gold marker, a tetracycline specific antibody-colloidal gold marker, a quinolone specific antibody-colloidal gold marker, a macrocyclic lipid specific antibody-colloidal gold marker, a sulfonamide specific antibody-colloidal gold marker, and a cephalosporin specific antibody-colloidal gold marker.

The reaction membrane may comprise a nitrocellulose membrane. The nitrocellulose membrane may be coated with which then coated with a macrolide antibiotics detection line, a beta lactam antibiotics detection line, a tetracyclines antibiotics detection line, a quinolones antibiotics detection line, a sulfonamides antibiotics detection line, a cephalosporin antibiotics detection line, and a goat anti mouse control line.

In some embodiments, the macrolide antibiotics detection line and the beta lactam antibiotics detection line are disposed in parallel of each other on the reaction membrane, the tetracyclines antibiotics detection line and the quinolones antibiotics detection line are disposed in parallel of each other on the reaction membrane, and the sulfonamides antibiotics detection line and the cephalosporin antibiotics detection line are disposed in parallel of each other on the reaction membrane.

In some embodiments, the macrolide antibiotics detection line comprises macrolide antibiotic-carrier protein, the beta lactam antibiotics detection line comprises beta lactam antibiotics-carrier protein, the tetracycline antibiotics detection line comprises tetracycline antibiotics-carrier protein, the quinolones antibiotics detection line comprises quinolone antibiotics-carrier protein the sulfonamide antibiotics detection line comprises sulfonamide antibiotics-carrier protein, and the cephalosporin antibiotics detection line comprises cephalosporin antibiotics-carrier protein. Each of the carrier protein may comprise bovine serum albumin.

Process for Preparing Colloidal Gold:

With double distilled deionized water to 5% mass fraction of chloroauric acid diluted into mass fraction to 0.01%, the heating rod magnetic stirrer to stir to boil, boil after each 100 ml gold chloride acid is added to the mass fraction of 5 ml 3% citric acid three sodium, the color of the solution turns from light yellow Bianlan color, then deep blue, finally to red and when the color of the solution completely into a transparent red, continue to back, after 10 min stop heating, cooling to room temperature with double distilled deionized water and added to the original volume of 4 DEG C. to avoid light preservation.

Preparation Method of Beta Lactam Antibiotic Specific Antibody Colloidal Gold Marker:

(1) By visual method to determine the colloidal gold labeled antibody and the ratio of potassium carbonate, with the regulation of 0.1 mol/L colloidal gold solution to 7.2, the antibody of pH 7.6, diluted 0.1 mol/L phosphate buffer by 5-40 g/ml, the 0.1 ml in order to join a series with 1 ml colloidal gold test tube, 5 min after the test tubes were added to the mass fraction of 0.1 ml 1% sodium chloride, mixing, static 2H observation results. From the test tube where the antibody concentration was 10 g/ml, the color of colloidal gold solution was basically the same, so the optimum anti body mass of 1 ml colloid was 10 mu g/ml.

(2) In magnetic agitation, in the 10 ml colloidal gold to add beta lactam antibiotics, special long antibody 10 ml, continue to stir, mix 10 min, add 0.2 ml 10% BSA, static 30 min. 4 C centrifugal 30 min, discarding the supernatant and precipitate with surfactant containing 0.1% mass fraction of bovine serum albumin, 0.5%, pH 0.2 mol/L 7.2 phosphate buffer washed two times, you can get the gold labeled antibody solution has been purified, 4 DEG C. and standby. Other gold labeled antibody solutions were prepared by the same method.

Sensitivity Test of Antibiotic Test Strip

Macrolide antibiotics, beta amide antibiotics, tetracyclines, quinolones, cefquinome were cephalosporins and sulfonamide antibiotics respectively with phosphate buffer pH 7 0.2 mol/L diluted to different concentrations, and then tested with the test paper of the invention, different concentrations of antibiotics to drop strip sample pad. The results show that the detection sensitivity of the test strip were: 10 g/L, erythromycin, spiramycin, penicillin 8 g/L G 4 g/L, amoxicillin 4 g/L, 15 g/L, oxytetracycline, chlortetracycline 30 g/L, 60 g/L, ofloxacin norfloxacin 100 g/L, 100 g/L, sulfadoxine sulfanilamide from Ta 100 g/L, head 20 Nobel wow mu g/L, head of Luoning 20 g/L.

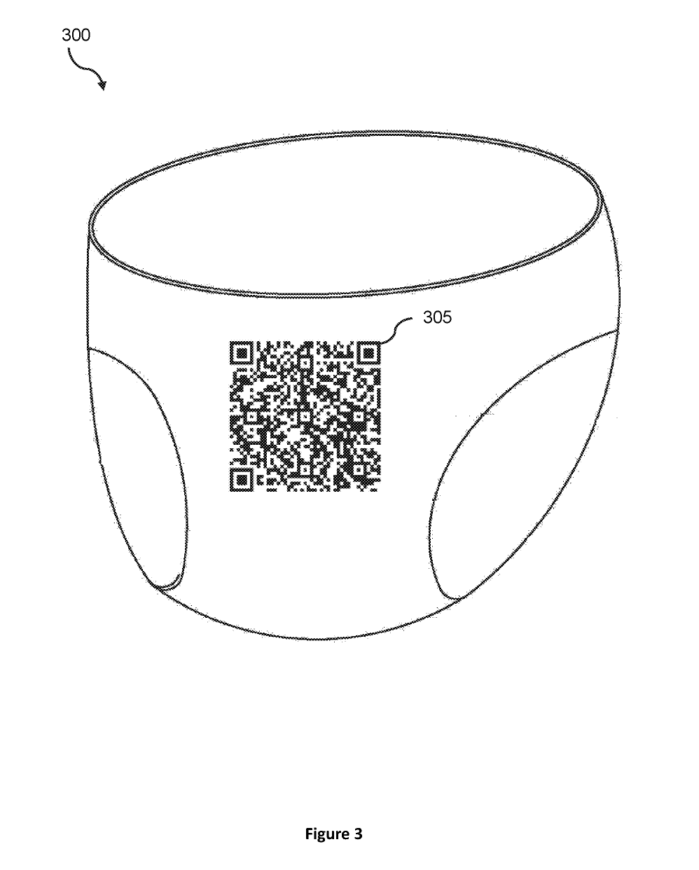

As shown in FIG. 3, an external sketch of a diaper 300 for children with urine detection function is provided. The diaper has a code 305 that can be scanned by a remote mobile terminal (not shown) for pairing between the remote mobile terminal and a urine detection device of the diaper 300.

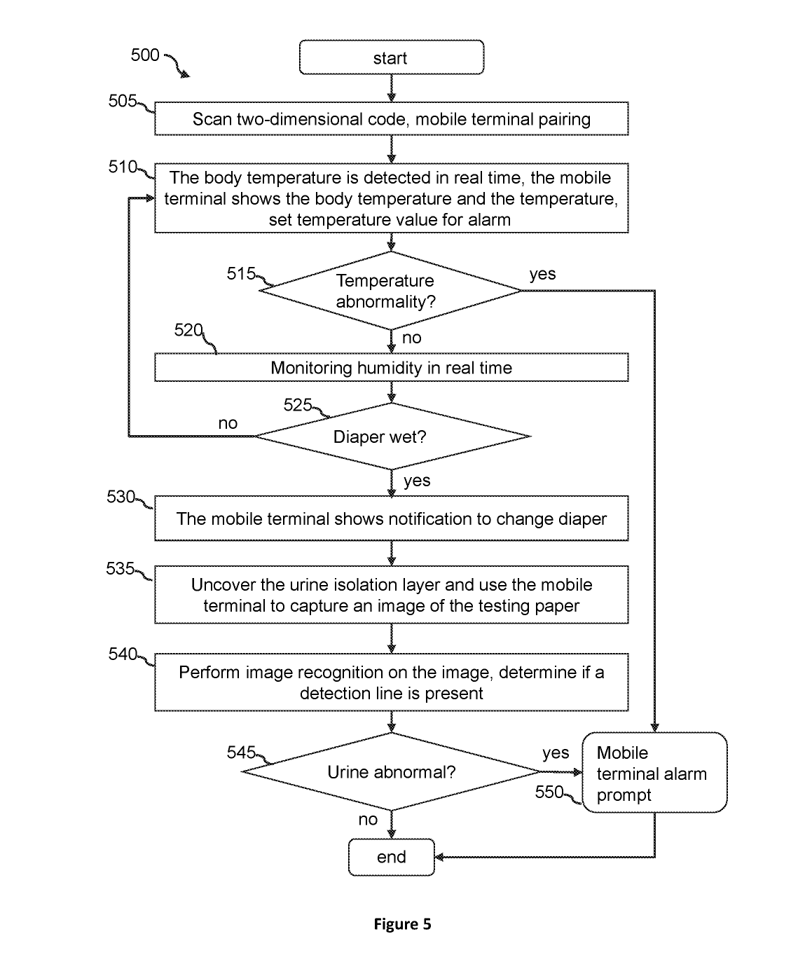

As shown in FIG. 5, the disclosure also provides a method for detection of antibiotics, urine detection device is arranged between the isolation layer in the urine in children's diapers and absorption core layer, wherein the urine detection device is connected with the wireless mobile terminal, including the detection step:

(1) In block 505, the mobile terminal scans the two-dimensional code on the diaper (an example of the code is shown as code 305 in FIG. 3) for pairing;

(2) In block 510, the urine detection device of the diaper detects the body temperature in real time, the mobile terminal displays the body temperature, sets the alarm value of the temperature, and when the temperature is determined to be abnormal (in block 515), carries out the alarm prompt by the mobile terminal in block 550. If the temperature is not abnormal, go to block 520;

(3) In block 520, monitor humidity in real time to determine whether diaper has wet urine, if it is determined to be wet (in block 525), go to block 530. If diaper is not wet, return block 510;

(4) in block 530, the mobile terminal prompts the user to change the diaper, and determine whether the antibiotic detection test paper of the detection device detects any abnormality in the urine collected in the diaper, comprising.

(4.1) In block 535, uncover the urine isolation layer and take an image of the test paper composite with the mobile terminal (as shown in FIG. 4);

(4.2) In block 540, perform an image recognition on the image of the test paper to detect if a detection line is present. The presence of one or more detection lines indicates an abnormality, while an absence of a detection line indicates a normal condition;

(4.3) In block 545, determine whether the test paper indicates abnormality in the urine, if it indicates abnormality, go to block 550 and the mobile terminal provides an alarm prompt.

The diapers according to various embodiments of the disclosure provide simple structure, low cost, can have a urine detection function, including temperature, trace elements, antibiotics, and routine urine test, so as to obtain users (children) of the health information, the mobile terminal can be combined with the use of long-term health monitoring of children.

The present disclosure, the accompanying figures, and the claims are not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure.

1. A diaper comprising:
a urine isolation layer;
a urine absorbent core layer disposed on the urine isolation layer;
an outer layer disposed on the urine absorbent core layer; and
a urine detection device disposed in between the urine isolation layer and the urine absorbent core layer,
wherein the urine isolation layer comprises an opening at one end of the urine isolation layer, the opening embedded in a sleeve of the absorption core layer.

2. The diaper of claim 1, wherein the urine detection device comprises a detection circuit and a test paper composite.

3. The diaper of claim 2, wherein the detection circuit comprises:
a controller;
a temperature sensor communicatively coupled to the controller and configured to obtain a temperature of a user;
a humidity sensor communicatively coupled to the controller and configured to detect a humidity of the absorbent core layer;
a wireless transmission module communicatively coupled to the controller and configured to communicate signals wirelessly with a remote mobile terminal; and
a power supply module configured to provide power to the controller, the temperature sensor, the humidity sensor, and the wireless transmission module.

4. The diaper of claim 3, wherein the wireless transmission module is operable in a Bluetooth wireless transmission mode and/or a WI-FI wireless transmission mode.

5. The diaper of claim 2, the test paper composite comprises a trace elements detection test paper, an antibiotic detection test paper, and a urine test strip.

6. The diaper of claim 5, wherein each of the trace elements detection test paper, the antibiotic detection test paper, and urine test strips comprises a plurality of test paper components, wherein each of the plurality of test paper components indicates a test result according to a different color indication scheme.

7. The diaper of claim 6, wherein each of the plurality of test paper components has a length between 5-30 mm, a width between 5-20 mm, and a thickness between 1-5 mm.

8. The diaper of claim 6, wherein the test paper components of the urine test strip detect at least a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

9. The diaper of claim 6, wherein the antibiotic detection test paper comprises a specimen absorbing pad, a specimen binding pad, a reaction membrane, and a liquid absorbing pad.

10. The diaper of claim 9, wherein the liquid absorbing pad comprises a filter paper.

11. The diaper of claim 9, wherein the specimen binding pad is coated with at least a beta lactam specific antibody-colloidal gold marker, a tetracycline specific antibody-colloidal gold marker, a quinolone specific antibody-colloidal gold marker, a macrocyclic lipid specific antibody-colloidal gold marker, a sulfonamide specific antibody-colloidal gold marker, and a cephalosporin specific antibody-colloidal gold marker.

12. The diaper of claim 9, wherein the reaction membrane comprises a nitrocellulose membrane coated with a macrolide antibiotics detection line, a beta lactam antibiotics detection line, a tetracyclines antibiotics detection line, a quinolones antibiotics detection line, a sulfonamides antibiotics detection line, a cephalosporin antibiotics detection line, and a goat anti mouse control line.

13. The diaper of claim 12, wherein the macrolide antibiotics detection line and the beta lactam antibiotics detection line are disposed in parallel of each other on the reaction membrane, wherein the tetracyclines antibiotics detection line and the quinolones antibiotics detection line are disposed in parallel of each other on the reaction membrane, and wherein the sulfonamides antibiotics detection line and the cephalosporin antibiotics detection line are disposed in parallel of each other on the reaction membrane.

14. The diaper of claim 12, wherein the macrolide antibiotics detection line comprises macrolide antibiotic-carrier protein, wherein the beta lactam antibiotics detection line comprises beta lactam antibiotics-carrier protein, wherein the tetracycline antibiotics detection line comprises tetracycline antibiotics-carrier protein, wherein the quinolones antibiotics detection line comprises quinolone antibiotics-carrier protein, wherein the sulfonamide antibiotics detection line comprises sulfonamide antibiotics-carrier protein, and wherein the cephalosporin antibiotics detection line comprises cephalosporin antibiotics-carrier protein.

15. The diaper of claim 14, wherein each of the carrier proteins comprises bovine serum albumin.

16. A method for detecting antibiotic elements in a diaper having a urine detection device communicatively coupled to a remote mobile terminal, comprising:
scanning, by the mobile terminal, a code on the diaper to perform a pairing between the mobile terminal and the urine detection device of the diaper;
monitoring, by the urine detection device, a humidity of the diaper based on a color change of a trace elements detection test paper of the diaper to determine if the diaper is wet; and
in response to a determination that the diaper is wet, providing, by the mobile terminal, a prompt for changing diapers.

17. The method of claim 16, further comprising:
determining, by the urine detection device, a temperature of the diaper, wherein the mobile terminal stores a normal temperature range; and
in response to a determination that the temperature of the diaper is an abnormal temperature based on the stored normal temperature range, providing, by the mobile terminal, an alert.

18. The method of claim 17, wherein determining the humidity of the diaper comprises:
removing a urine isolation layer of the diaper and capturing, by the mobile terminal, an image of the trace elements detection test paper;
detecting, by the mobile terminal, a color indication of the trace elements detection test paper;
determining, by the mobile terminal, an abnormal condition based on a detection of a detection line on the diaper; and
providing, by the mobile terminal, an alert in response to determining the abnormal condition.
